(19) United States
(12) Patent Application Publication
(10) Pub. No.: US 2022/0042953 A1
(43) Pub. Date: Feb. 10, 2022

(54) SYSTEM AND METHOD FOR REDUCING MOISTURE TO SAMPLE AND TEST A GAS MIXTURE

(71) Applicant: Computational International LLC, Newark, DE (US)

(72) Inventors: Ken Pingsheng HU, Newark, DE (US); Geoffrey Stephen ENGEL, Souderton, PA (US); Drew Gentner, New York, NY (US)

(21) Appl. No.: 17/397,599

(22) Filed: Aug. 9, 2021

Related U.S. Application Data
(60) Provisional application No. 63/063,883, filed on Aug. 10, 2020.

(51) Int. Cl. G01N 30/14; G01N 33/00; G01N 30/72; G01N 30/74

(52) U.S. Cl. CPC G01N 30/14; G01N 30/74; G01N 30/7206; G01N 33/0047

(57) ABSTRACT

A system for analyzing a gas mixture is provided. The system includes an enclosure inlet. A moisture trap assembly is coupled to the enclosure inlet. The moisture trap assembly removes excess moisture from a sample at the enclosure inlet. A testing section is coupled to the moisture trap assembly for detecting one or more compounds from the sample.

### CROSS REFERENCE TO RELATED APPLICATIONS

This application claims priority to U.S. provisional application No. 63/063,883 filed on Aug. 10, 2020, the contents of which is included herein in its entirety.

### BACKGROUND

Sampling and testing analytes in gas and air for volatile organic compounds (VOCs) is critical for business, government as well as consumers. VOC testing instruments can be used in a wide range of applications, such as testing of human breath for disease diagnostic and screening purposes, testing of air and/or gas for VOCs in a high temperature and high humidity setting for air pollution detection purpose, testing of solid and/or liquid such as juice, milk, medicine for food and drug safety purposes.

VOC testing instruments are impaired by their ability to performing testing for analytes in high humidity gas or air. While a number of systems can effectively remove humidity, for example by passing the sample to be tested through activated carbon pellets, these systems also remove target analytes, such as VOCs. This impairs these instruments capabilities and can defeat the purpose of testing all together.

Other methods for the removal of humidity use high purity carrier gases with low or no humidity to purge the VOC testing instrument. However, such methods are expensive, and not practical for operation in a non-laboratory application or in a home. Without the use of pure and no humidity carrier gases the current VOC testing methodologies are unable to perform to ultra-sensitive analysis of high humidity test samples.

It is desirable for VOC testing equipment to detect levels of VOCs at Parts-Per-Trillion (ppt), without the need for pure and no humidity carried gases. The removal of humidity from the sampled gas and/or air should not remove extremely low levels of VOCs, which would defeat the purpose of the testing.

### BRIEF SUMMARY

According to one aspect of the subject matter described in this disclosure, a system for analyzing a gas mixture is provided. The system includes an enclosure inlet. A moisture trap assembly is coupled to the enclosure inlet. The moisture trap assembly removes excess moisture from a sample at the enclosure inlet. A testing section is coupled to the moisture trap assembly, the testing section includes a plurality of valves, and a plurality of sensors being coupled to at least one of the valves. The sensors detect inorganic and organic chemicals from the sample.

According to another aspect of the subject matter described in this disclosure, a system for analyzing a gas mixture is provided. The system includes an enclosure inlet. A moisture trap assembly is coupled to the enclosure inlet. The moisture trap assembly removes excess moisture from a sample at the enclosure inlet. A testing section is coupled to the moisture trap assembly for detecting one or more compounds from the sample.

According to another aspect of the subject matter described in this disclosure, a method for analyzing a gas mixture is provided. The method includes providing an enclosure inlet. Also, the method includes coupling a moisture trap assembly to the enclosure inlet. The moisture trap assembly removes excess moisture from a sample at the enclosure inlet using a moisture trap tube. Moreover, the method includes detecting, using a testing section coupled to the moisture trap assembly, one or more compounds from the sample.

Additional features and advantages of the present invention are described in, and will be apparent from, the detailed description of this disclosure.

### BRIEF DESCRIPTION OF THE DRAWINGS

The disclosure is illustrated by way of example, and not by way of limitation, in the figures of the accompanying drawings in which like reference numerals are used to refer to similar elements. It is emphasized that various features may not be drawn to scale and the dimensions of various features may be arbitrarily increased or reduced for clarity of discussion.

FIG. 1 is a schematic diagram illustrating a top view of a testing system, in accordance with some embodiments.

FIG. 2 is a schematic diagram illustrating an exploded view of an exemplary embodiment of an oven assembly, in accordance with some embodiments.

FIG. 3 is a schematic diagram illustrating an exploded view of an exemplary embodiment of a GC trap assembly, in accordance with some embodiments.

FIG. 4 is a schematic diagram illustrating an exploded view of an exemplary embodiment of a moisture trap assembly, in accordance with some embodiments.

FIG. 5 is a schematic diagram illustrating an exemplary embodiment of a gas chromatography (GC) Trap tube assembly, in accordance with some embodiments.

FIG. 6 is a schematic diagram illustrating an exemplary embodiment of a moisture trap tube, in accordance with some embodiments.

FIG. 7 is a schematic diagram illustrating an exemplary embodiment of a combine filter assembly, in accordance with some embodiments.

FIG. 8 is a schematic diagram illustrating an exemplary embodiment of a moisture trap assembly used in conjunction with a standalone system, in accordance with some embodiments.

FIG. 9 is a schematic diagram illustrating an exemplary embodiment of an enclosure for a VOC testing system, in accordance with some embodiments.

FIG. 10 is a schematic diagram illustrating an exemplary embodiment of a testing arrangement, in accordance with some embodiments.

### DETAILED DESCRIPTION

The figures and descriptions provided herein may have been simplified to illustrate aspects that are relevant for a clear understanding of the herein described devices, systems, and methods, while eliminating, for the purpose of clarity, other aspects that may be found in typical similar devices, systems, and methods. Those of ordinary skill may recognize that other elements and/or operations may be desirable and/or necessary to implement the devices, systems, and methods described herein. But because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

The terminology used herein is for the purpose of describing particular example embodiments only and is not intended to be limiting. For example, as used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. That is, terms such as “first,” “second,” and other numerical terms, when used herein, do not imply a sequence or order unless clearly indicated by the context.

A VOC testing system is described herein that uses a moisture trap assembly in conjunction with multiple other systems to form a VOC testing system. The VOC testing system may enable ultra-sensitive VOC testing in high moisture situations while achieving high analyte sensitivity as well as selectivity. By way of a non-limiting example, such a system can identify and separate a wide range of VOCs in low concentrations in a high humidity situation.

In some embodiments, a system may use the moisture trap assembly with a non-VOC testing system. In this case, the moisture trap assembly may be used in conjunction with any system requiring moisture removal while leaving target analytes intact, whether VOC or not. By way of a non-limiting example, the moisture trap assembly may be used in conjunction with another system that can detect particulate matters, droplets, and other analytes, such as SO₂, NH4, or the like.

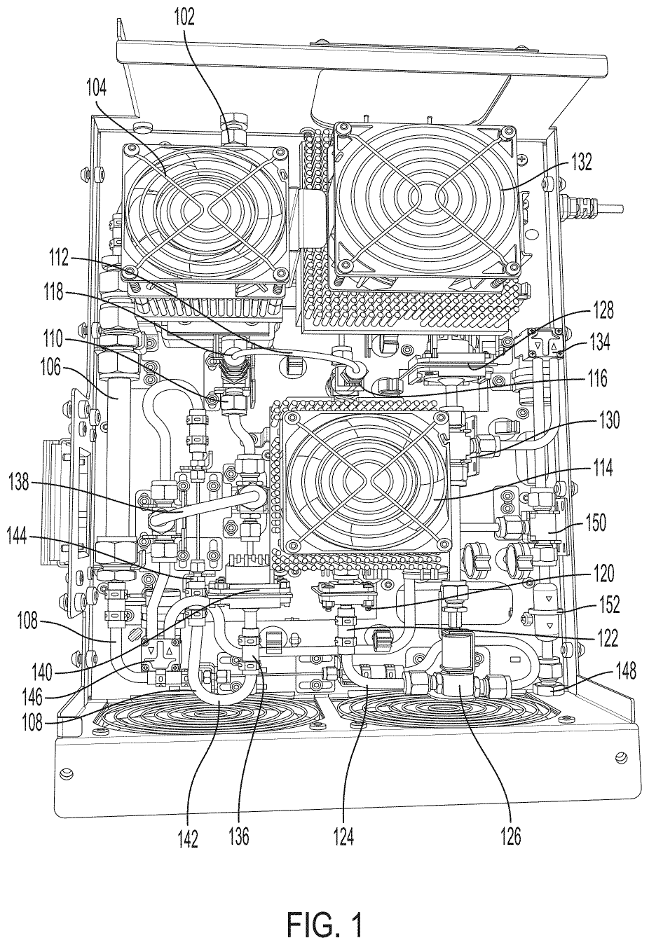

FIG. 1 shows a top view of a VOC analyte testing system 100, in accordance with some embodiments. Testing system 100 may include an enclosure inlet being connected to tee 102. Tee 102 may be connected to a moisture trap assembly 104 and a combined filter assembly 106. The moisture trap assembly 104 may include a moisture trap tube. The combined filter assembly 106 may include a carbon filter and a water filter. The combined filter assembly 106 may be connected to a special purpose tubing 108. The moisture trap assembly 104 may be connected to tee 110, which contains the sampling tube 112 to draw air and/or gases towards the GC Trap assembly 114. Also, tee 110 may be connected to control valve 116 and connected to control valve 118.

Control Valve 116 may also be connected to oven and GC Trap assembly 114. GC Trap assembly 114 may be connected to pre-trap 120. Pre-trap 120 may be connected to control valve 122. Control valve 122 may be connected to special purpose tubing 124 and tee 126. GC photoionisation detector (PID) sensor 128 may be connected to tee 130 and oven 132. Also, GC PID sensor 128 may be used for GC VOC testing. Tee 130 may be connected to GC Pump 134. Control valve 136 may be connected to tee 138, and connected to total volatile organic compound (TVOC) PID sensor 140. TVOC PID sensor 140 may be used for TVOC testing. TVOC PID sensor 140 may be connected to TVOC special purpose tubing 142. TVOC special purpose tubing 142 may be connected to tee 144. Tee 138 may be connected to TVOC Pump 146. GC Pump 134 and TVOC Pump 146 may be connected to enclosure outlet 148.

In this implementation, the enclosure outlet 148 may include a tee 150 that may be connected to a muffler 152. Muffler 152 may be connected to outlet 148. Outlet 148 may be used to expel gas, air, and/or exhaust from testing system 100.

In some embodiments, the number of interconnections used in testing system 100 between components and/or placement of the components may change to meet particular size requirements of the enclosure. In some embodiments, the number of tees used in testing system 100 may be different and/or arranged differently as shown in FIG. 1.

In some embodiments, the GC PID sensor 128 may be a micro-electromechanical systems (MEMS) sensor or a mass spectrometer performing GC VOC testing.

In some embodiments, the TVOC PID sensor 140 may be a MEMS sensor performing TVOC testing.

In some embodiments, the testing system 100 may include at least one moisture sensor, temperature sensor, or dark matter count sensor for testing.

In some embodiments, the GC PID sensor 128 or TVOC PID sensor 146 may be used to detect inorganic and organic chemicals from the sample.

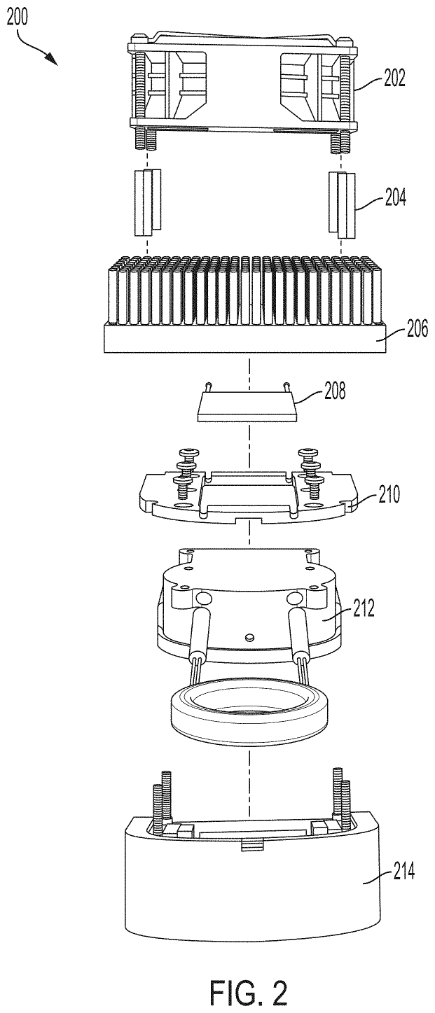

FIG. 2 shows an exploded view of an exemplary embodiment of an oven assembly 200, in accordance with some embodiments. The oven assembly 200 may include a cooling fan 202 used to cool the oven assembly 200. A heatsink 206 may be positioned beneath the cooling fan 202 to remove heat from the oven assembly 200.

Several cooling devices 204 may be used to provide additional cooling to oven assembly 200 and heatsink 206. Also, cooling devices 208 may be positioned in the middle of an insulation spacer 210. The insulation spacer 210 may be retained to the oven assembly 200 using screws. A toroid tubing may be placed inside of oven housing 212. Thermal insulation material may be used to insulate the oven housing 212 from the ambient air and thereby retain the heat or cooling applied to the oven housing 212. Oven assembly 200 may include an enclosure 214 having screws to hold the entire oven assembly 200 together.

In some embodiments, oven assembly 200 may incorporate heating elements and cooling devices so that it can control the temperature from −10 degC to 220 degC. Moreover, oven assembly may incorporate slots to hold the tubing interconnections.

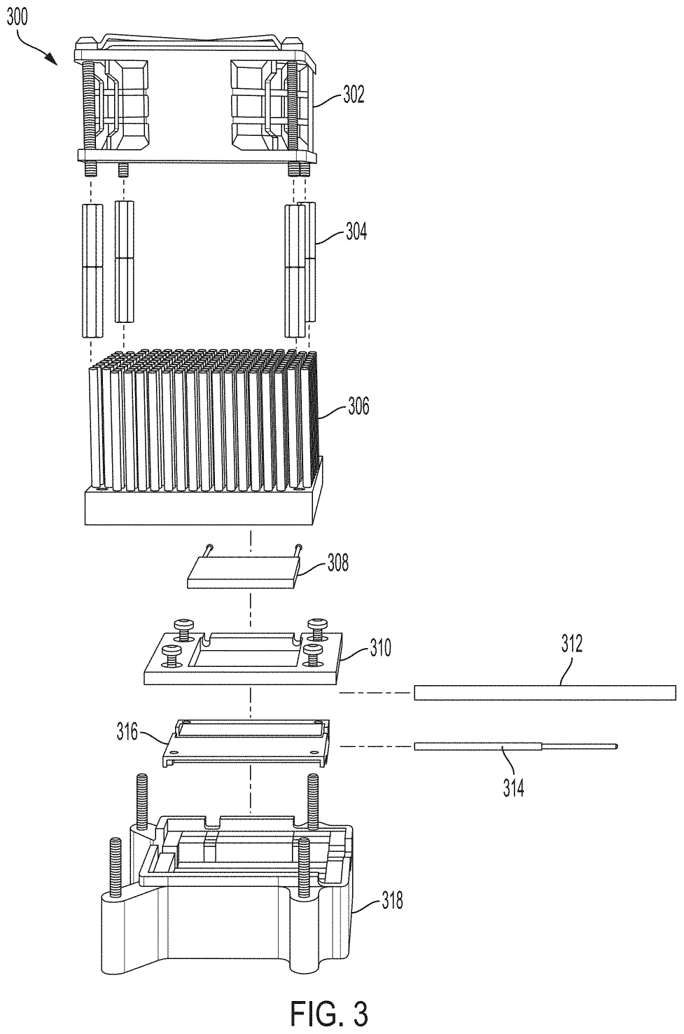

FIG. 3 illustrates an exploded view of an exemplary embodiment of a GC trap assembly 300, in accordance with some embodiments. The GC trap assembly 300 may include a cooling fan 302 used to cool GC trap assembly 300. A heatsink 306 may be positioned beneath the cooling fan 302 to remove heat from GC trap assembly 300. A spacer 308 may be positioned beneath heatsink 306. Several cooling devices 304 may be positioned on either side of cooling fan 302 to assist with heat removal. Screws 310 may retain the spacer 308 to the GC Trap assembly 300. Thermal insulation may be included into spacer 308.

A GC trap tube 312 may be positioned in the GC trap assembly 300. GC Trap assembly may include heating elements 314. GC Trap housing 316 may be positioned in thermal insulation material which is used to insulate the GC Trap housing 316 from the ambient air and thereby retaining the heat or cooling applied to the GC Trap housing 316.

In some embodiments, GC Trap assembly 300 may utilize heating elements cooling device to control the temperature of the GC Trap from −10 degC to 220 degC. GC Trap assembly 300 may include a GC Trap tube.

In some embodiments, enclosure 318 may enclose GC trap assembly 300 within thermal insulation to assist in the control of the temperature of GC trap assembly 300. This insulation may surround GC trap assembly 300.

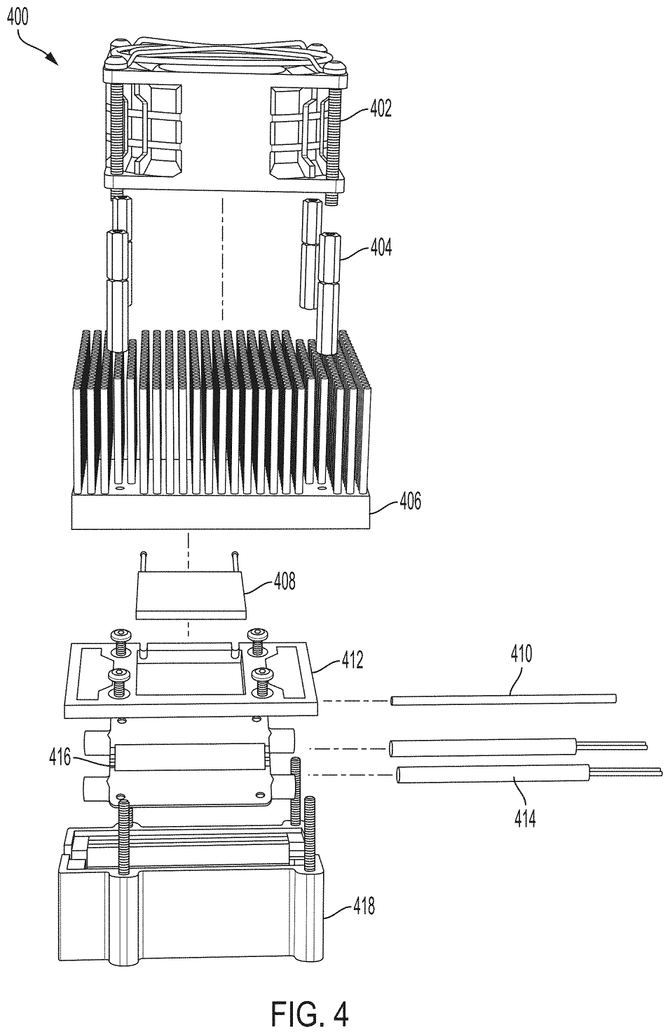

FIG. 4 illustrates an exploded view of an exemplary embodiment of a moisture trap assembly 400, in accordance with some embodiments. The moisture trap assembly 400 may include a cooling fan 402 used to cool moisture trap assembly 400. A heatsink 406 may be positioned beneath the cooling fan 402 to remove heat from moisture trap assembly 400. A spacer 408 may be positioned beneath heatsink 406. Several cooling devices 404 may be positioned to assist with heat removal. Screws 412 may retain the spacer 408 to the moisture trap housing 416. Moisture trap housing 416 may include heating elements 414. Also, moisture trap housing 416 may be positioned in thermal insulation material, which is used to insulate the moisture trap housing 416 from the ambient air and thereby retain the heat or cooling applied to the moisture trap assembly 400. The moisture trap housing 416 and clamp assembly 410 may be positioned in an enclosure 418. Enclosure 418 may include screws to hold the entire moisture trap assembly 400 together.

In some embodiments, enclosure 418 may enclose moisture trap assembly 400 within a thermal insulation to assist in the control of the temperature of moisture trap assembly 400. This insulation may surround moisture trap assembly 400.

In some embodiments, moisture trap assembly 400 may utilize heating elements and a cooling devices to control the temperature of the moisture trap assembly 400 from −10 degC to 150 degC. Moisture trap assembly 400 may include a moisture trap tube, which is described further hereinafter.

In some embodiments, the sampling of air and/or gases that have passed through the moisture trap assembly may need to be done in a way that ensures that certain type of moisture is not drawn through to the GC Trap assembly including but not limited to an example such as GC Trap assembly 300 described above. The use of a sampling tube that draws gases from the center of the tubing after the moisture trap may be used. The sampling tube may be manufactured from an inert material so as to not affect the sampled air and/or gases and allow manipulation of moisture inside of the sampling tube. The efficiency of the sampling tube will depend upon the length, position, internal diameter and other factors known to the skilled artisan.

In some embodiments, heating may be achieved with heater which may require a heating controller. Heating control may be capable of increasing the moisture trap temperature to a suitable level to allow the heat and airflow to disperse the moisture. Moisture trap assembly 400 may include heater element(s) that may be controlled by a continuously variable voltage.

In some embodiments, the temperature of moisture trap assembly 400 may be controlled with heating and cooling. This temperature may be measured and controlled (via heating and cooling) in a refined manner.

In some embodiments, the moisture trap assembly 400 may have its own power source which it may be able to operate independently from a system such as testing system 100.

In some embodiments, the moisture trap assembly 400 may be an independent system having a plurality of pumps and/or valves, inlets and outlets. Also, the moisture trap assembly 400 may include a plurality of connectors, such as tees or the like.

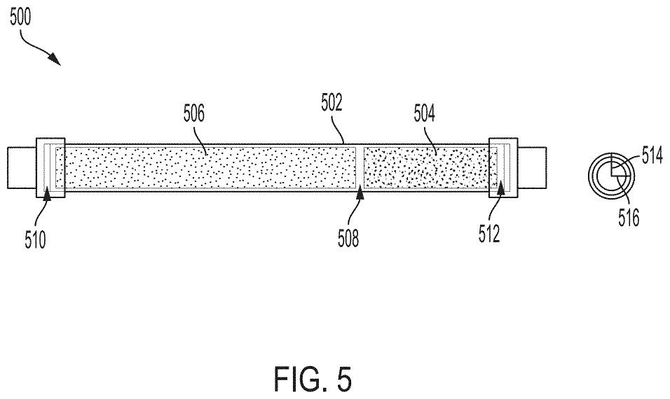

FIG. 5 illustrates an exemplary embodiment of a GC Trap tube assembly 500, in accordance with some embodiments. The GC Trap tube assembly 500 may include a tube 502, such as a stainless-steel tube or the like, that is designed to concentrate and/or trap and then release of analytes such as VOCs. The GC Trap tube assembly 500 may include different types of absorbent and/or adsorbent materials 504 and 506 with different characteristics which are retained in the tube 502. These different absorbent and/or adsorbent materials 504 and 506 may be mixed or in some cases separated by specially made separation materials 508 in different compartments in the tube 502. At both ends of the tube 502, containment apparatuses 510 and 512 may be used to enclose the absorbent and/or adsorbent materials 504 and 506.

In some embodiments, GC Trap tube assembly 500 may include an outside diameter 514 of 1 mm up to 80 mm depends on the application. The internal diameter 516 may depend on the absorbent materials used which can vary from 0.5 mm up to 78 mm.

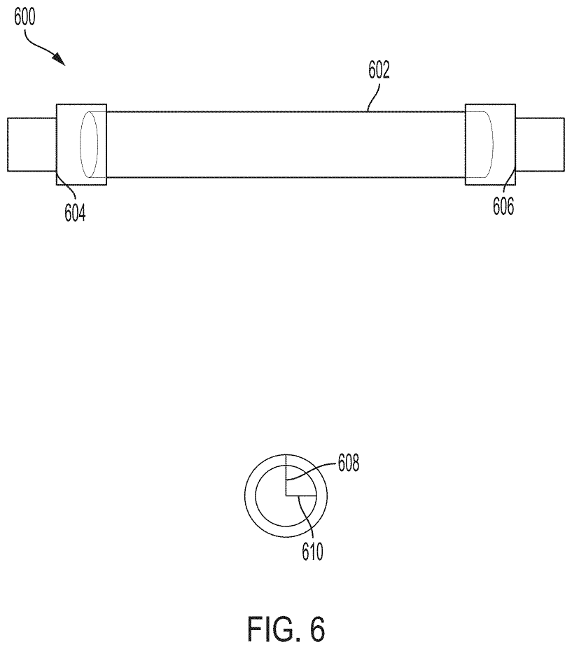

FIG. 6 shows an exemplary embodiment of a moisture trap tube 600, in accordance with some embodiments. The moisture trap tube 600 may include a tube 602 that is used to perform the removal of moisture and/or water molecule without affecting the gasses and/or analytes such as VOCs being passed through it. The moisture trap tube 602 may contain materials to facilitate the removal of moisture, or no material inside the moisture trap tube 600. Tube containments 604 and 606 may be connected on both ends of moisture trap assembly 600. The moisture trap assembly 400 may work in conjunction with a moisture trap tube 600 to remove moisture from a sample.

In some embodiments, moisture trap tube 600 may have an outside diameter 608 of 1 mm to 100 mm with an inside diameter 610 of 0.5 mm to 98 mm depending on the moisture removal material and/or method used.

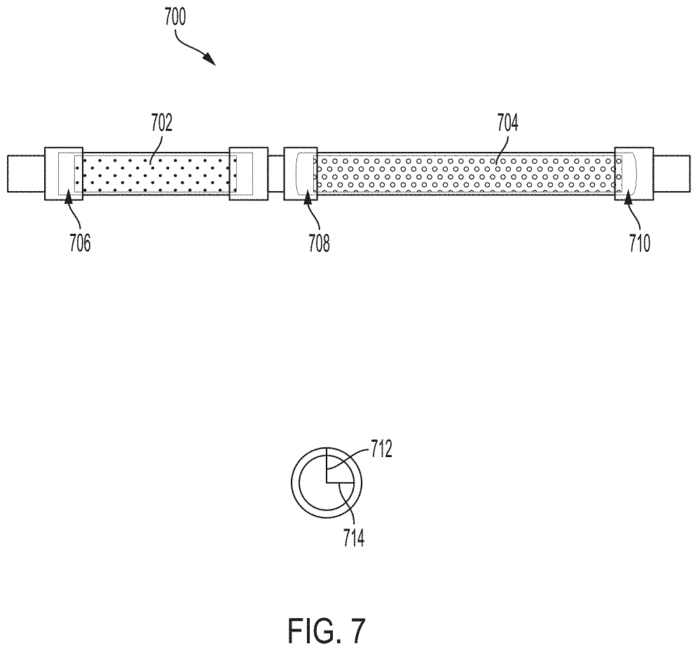

FIG. 7 shows an exemplary embodiment of a combined filter assembly 700, in accordance with some embodiments. The combined filter assembly 700 may include a first tube portion 702 that serves to filter out undesirable gases from the gas and/or air drawn through it, and second tube portion 704 to filter out moisture and/or water. The combined filter assembly 700 integrates at least water and carbon trapping materials in the first tube portion 702 and the second tube portion 704 including but not limited to activated carbon which are known to the skilled artisan. The materials inside the combined filter assembly 700 may be retained through the use of porous apparatus which permit airflow without the loss of the contained materials. The materials used in the combined filter 700 may be inert so as not to affect the testing of the airflow drawn through the combined filter assembly.

Tube containments 706 and 710 may be used at the ends of combine filter assembly 700. Also, tube containment 708 may be used to connect first tube portion 702 and second tube portion 704.

In some embodiments, the first tube portion 702 and the second tube portion 704 may include stainless steel.

The combined filter tube 700 may have an outside diameter 712 of 1 mm to 100 mm while an inside diameter 714 of 0.5 mm to 98 mm depending on the moisture removal material and/or method.

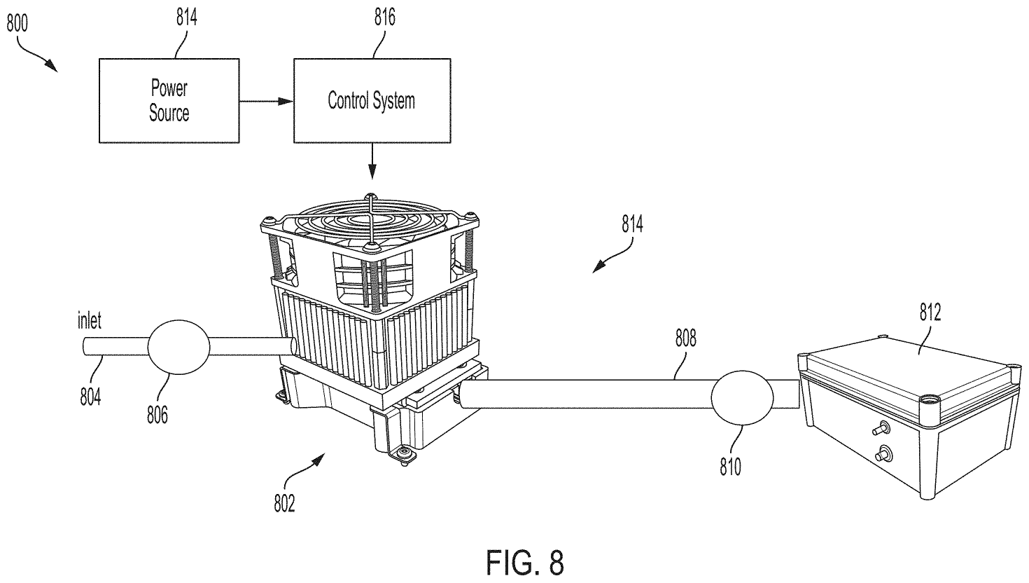

FIG. 8 shows an exemplary embodiment of a moisture trap assembly 800 used in conjunction with a standalone testing system, in accordance with some embodiments. The moisture trap assembly 802 may operate with a standalone system 812, which requires moisture removal while preserving targeted analytes including but not limited to VOCs. In this case, the standalone system 812 may be a non-VOC testing system that requires moisture removal. The moisture trap assembly 802 may receive the targeted analytes via inlet 804. The inlet 804 may be coupled to a first control arrangement 806 having a plurality of pumps, control valves, and/or connectors. The output of moisture trap assembly 800 may be connected to the standalone system via an outlet 808. The outlet 808 may be coupled to a second control arrangement 810 having a plurality of pumps, control valves, and/or connectors.

The moisture trap assembly 800 may be connected to power source elements 814, 816 for powering moisture trap assembly 800. Power source element 814 may be a power supply/power source. Power source element 816 may be a control system coupled to power source element 814 to control the power provided by power source element 814 to moisture trap assembly 800.

In some embodiments, the standalone system 812 may be used to detect inorganic and organic chemicals from a sample.

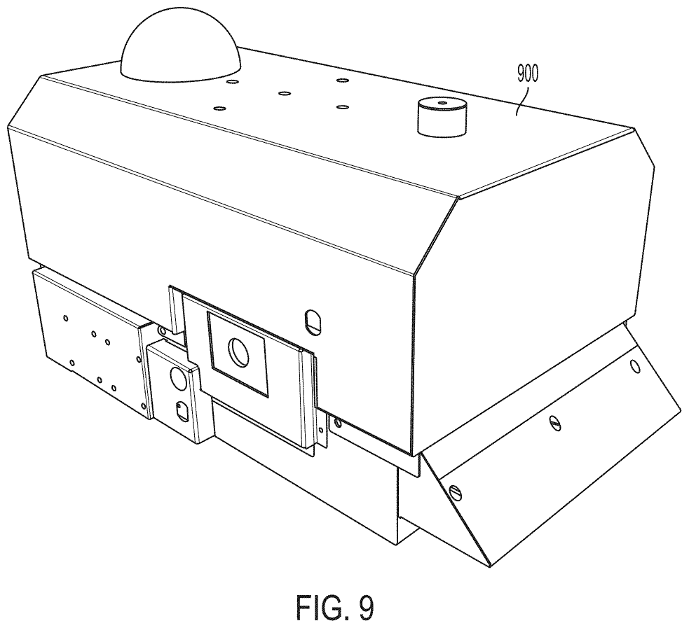

FIG. 9 shows an exemplary embodiment of an enclosure 900 for a VOC testing system, in accordance with some embodiments. Enclosure 900 may be water resistant, dust resistant, anti-theft, anti-impact, and highly resilient. The enclosure 900 may protect the VOC testing system while enabling the functionality of a moisture trap. In some embodiments, the VOC testing may include testing system 100.

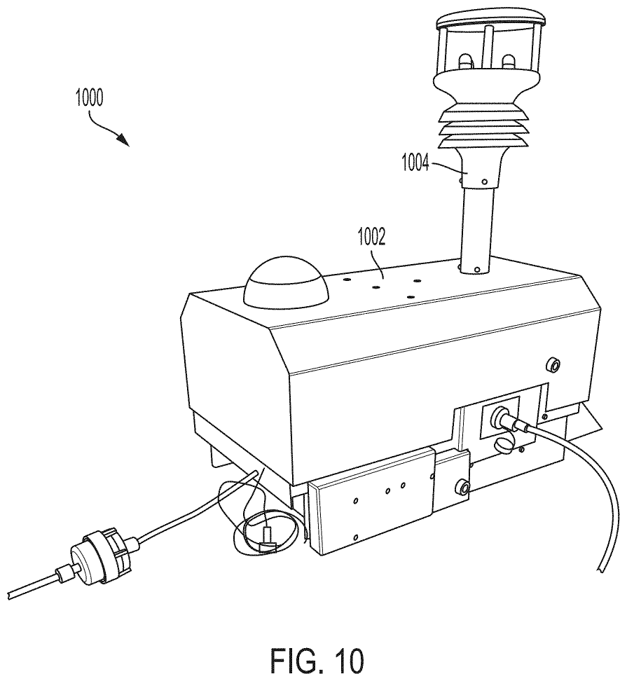

FIG. 10 shows an exemplary embodiment of a testing arrangement 1000, in accordance with some embodiments. The testing arrangement 1000 may include a communication antenna and a wind meter unit 1004 integrated into a testing system 1002. In some embodiments, the VOC testing may include testing system 100.

In some embodiments, testing arrangement 1000 may include key access to data storage such as a SD card, external display such as a LCD/LED, water and dust resistant structures, low air flow resistance inlet and exhaust structures, and anti-theft, anti-interference, and shock and impact resistant structures.

In some embodiments, testing system 1002 may include an Internet of thing (IoT) connected VOC testing system which also uses the external antenna and the wind meter 1004.

The disclosure describes a moisture trap assembly to be used in conjunction with an ultra-sensitive VOC testing system or non-VOC testing system. The disclosure describes an internal structure which allows the moisture trap assembly to be mounted and integrated as part of the ultra-sensitive VOC testing system. This system can identify and separate a wide range of VOCs in low concentrations in a high humidity environment.

Also, the disclosure describes an arrangement that allows the moisture trap assembly to operate as a standalone system working in conjunction with a non-VOC testing system. This system may be used to detect particulate matters, droplets, and other analytes, such as SO₂, NH₄, or the like.

Reference in the specification to “one implementation” or “an implementation” means that a particular feature, structure, or characteristic described in connection with the implementation is included in at least one implementation of the disclosure. The appearances of the phrase “in one implementation,” “in some implementations,” “in one instance,” “in some instances,” “in one case,” “in some cases,” “in one embodiment,” or “in some embodiments” in various places in the specification are not necessarily all referring to the same implementation or embodiment.

Finally, the above descriptions of the implementations of the present disclosure have been presented for the purposes of illustration and description. It is not intended to be exhaustive or to limit the present disclosure to the precise form disclosed. Many modifications and variations are possible in light of the above teaching. It is intended that the scope of the present disclosure be limited not by this detailed description, but rather by the claims of this application. As will be understood by those familiar with the art, the present disclosure may be embodied in other specific forms without departing from the spirit or essential characteristics thereof. Accordingly, the present disclosure is intended to be illustrative, but not limiting, of the scope of the present disclosure, which is set forth in the following claims.

What is claimed is:

1. A system for analyzing a gas mixture comprising:
an enclosure inlet;
a moisture trap assembly coupled to the enclosure inlet, wherein the moisture trap assembly removes excess moisture from a sample at the enclosure inlet; and
a testing section coupled to the moisture trap assembly, the testing section comprising:
a plurality of valves; and
a plurality of sensors being coupled to at least one of the valves, wherein the sensors detect inorganic and organic chemicals from the sample.

2. The system of claim 1, wherein the testing section comprises an oven coupled to one of the valves and a first sensor of the plurality of sensors.

3. The system of claim 2, wherein the first sensor performs gas chromatography (GC) VOC testing for both organic and inorganic testing.

4. The VOC testing system of claim 2, wherein the first sensor is a photoionisation detector (PID) sensor, a micro-electromechanical systems (MEMS) sensor, or a mass spectrometer.

5. The VOC testing system of claim 3, wherein the sensors comprise at least one photoionisation detector (PID) or micro-electromechanical systems (MEMS) sensor performing a total volatile organic compound (TVOC) testing.

6. The system of claim 3 further comprising a filter arrangement coupled to the testing section.

7. A system for analyzing a gas mixture comprising:
an enclosure inlet;
a moisture trap assembly coupled to the enclosure inlet, wherein the moisture trap assembly removes excess moisture from a sample at the enclosure inlet; and
a testing section coupled to the moisture trap assembly for detecting one or more compounds from the sample.

8. The system of claim 8, wherein the testing section is a non-VOC testing system.

9. The system of claim 8, wherein the testing section is a volatile organic compound (VOC) testing system.

10. The system of claim 9 further comprising an oven coupled to one of the valves and a first sensor of the plurality of sensors.

11. The system of claim 9, wherein the first sensor performs gas chromatography (GC) VOC testing.

12. The system of claim 11, wherein the first sensor is a photoionisation detector (PID) sensor, a micro-electromechanical systems (MEMS) sensor, or a mass spectrometer.

13. The system of claim 9, wherein the sensors comprise at least one total volatile organic compound (TVOC) sensor performing TVOC testing.

14. A method for analyzing a gas mixture comprising:
providing an enclosure inlet;
coupling a moisture trap assembly to the enclosure inlet, wherein the moisture trap assembly removes excess moisture from a sample at the enclosure inlet using a moisture trap tube; and
detecting, using a testing section coupled to the moisture trap assembly, one or more compounds from the sample.

15. The method of claim 14, wherein the testing section is a non-VOC testing system.

16. The method of claim 14, wherein the testing section is a volatile organic compound (VOC) testing system.

17. The system of claim 16 further comprising an oven coupled to one of the valves and a first sensor of the plurality of sensors.

18. The system of claim 16, wherein the first sensor performs gas chromatography (GC)-based testing.

19. The system of claim 18, wherein the first sensor is a photoionisation detector (PID) sensor, a micro-electromechanical systems (MEMS) sensor, or mass spectrometer.

20. The system of claim 16, wherein the sensors comprise at least one total moisture sensor, temperature sensor, dark matter count sensor, or a total volatile organic compound (TVOC) sensor.
